(19) United States
(12) Patent Application Publication
(10) Pub. No.: US 2024/0101941 A1
(43) Pub. Date: Mar. 28, 2024

(54) Bioreactor Apparatus and Related Methods for Converting Carbon Dioxide to Oxygen

(71) Applicant: Nigel Salvador Pereira, Scarsdale, NY (US)

(72) Inventor: Nigel Salvador Pereira, Scarsdale, NY (US)

(21) Appl. No.: 17/953,574

(22) Filed: Sep. 27, 2022

(51) Int. Cl. C12M 1/00; C12M 1/107; C12M 1/34; C12N 1/12; C12P 3/00

(52) U.S. Cl. CPC C12M 21/02; C12M 23/34; C12M 23/36; C12M 29/04; C12M 31/10; C12M 41/30; C12N 1/12; C12P 3/00

(57) ABSTRACT

The present disclosure provides a bioreactor that utilizes cultivated algae grown within a sealed chamber to convert carbon dioxide in fluid that is passed through the sealed chamber into oxygen via photosynthesis. The apparatus comprises an LED array for providing artificial light to the algae, which causes them to utilize CO2 in the surrounding fluid for photosynthesis, producing oxygen as a result. Transparent inserts are disposed within the sealed chamber for increasing the amount of surface area on which the algae can form and increasing efficiency.

### FIELD OF INVENTION

The present invention relates generally to methods and apparatus for carbon capture and conversion to oxygen. More specifically, the present invention relates to a bioreactor apparatus and methods of using algae cultivated therein to convert carbon dioxide to oxygen.

### BACKGROUND

In the present day there is a massive global issue with the excessive amount of carbon dioxide in the atmosphere. Estimates have been made that Earth's atmosphere has accumulated over 1.5 trillion tons of carbon dioxide since 1751. These estimates can be seen as an accumulation of all greenhouse contributors. And because transportation vehicles contribute up to 78% of these 5,000 tons of carbon dioxide, controlling them is of primary concern.

With the ever-decreasing amount of free space due to the expansion of cities, an alternative is required to simply planting trees. The process is simply not fast enough—in order to effectively decrease carbon dioxide, trees have to be fully grown, which can take up to 4 years.

Photosynthesis splits water to liberate O₂ and transforms CO₂ into sugar. Photosynthetic organisms are photoautotrophs, which means that they are able to synthesize food directly from carbon dioxide using energy from light. However, not all organisms that use light as a source of energy carry out photosynthesis, since photoheterotrophs use organic compounds, rather than carbon dioxide, as a source of carbon. In plants, algae and cyanobacteria, photosynthesis releases oxygen. This is called oxygenic photosynthesis. Although there are some differences between oxygenic photosynthesis in plants, algae and cyanobacteria, the overall process is quite similar in these organisms.

Commercial and industrial algae cultivation is already a thriving industry and has numerous uses, including production of food ingredients, food, fertilizer, bioplastics, dyes and colorants, chemical feedstock, pharmaceuticals, and algal fuel.

An additional benefit proposed by the present application is that the use of algae can help to stabilize the concentration of carbon dioxide in the atmosphere, because during photosynthesis, algae and other photosynthetic organisms capture carbon dioxide and sunlight and convert those into oxygen and biomass. Water, carbon dioxide, minerals and light are all important factors in algae cultivation, and different algae have different requirements, but if algae are chosen specifically for their carbon capture efficiency then low power carbon capture bioreactors could go a long way towards solving the carbon crisis.

It is within this context that the present invention is provided.

### SUMMARY

The present disclosure provides a bioreactor that utilizes cultivated algae grown within a sealed chamber to convert carbon dioxide in fluid that is passed through the sealed chamber into oxygen via photosynthesis. The apparatus comprises an LED array for providing artificial light to the algae, which causes them to utilize CO₂ in the surrounding fluid for photosynthesis, producing oxygen as a result. Transparent inserts are disposed within the sealed chamber for increasing the amount of surface area on which the algae can form and increasing efficiency.

According to a first aspect of the present disclosure, there is provided a bioreactor apparatus for converting Carbon Dioxide to Oxygen, the apparatus comprising: a sealed chamber, the chamber comprising one or more walls having one or more inner surfaces; one or more planar transparent inserts disposed within the sealed chamber, the surfaces of the one or more inserts being suitable for hosting algae; an LED array disposed on a portion of the one or more inner surfaces of the sealed chamber, the LED array being configured to emit light suitable for causing photosynthesis in algae; a power source connected to the LED array; an inlet configured to deliver a flow of fluid containing Carbon Dioxide into the chamber; and an outlet configured to release Oxygen from the sealed chamber.

In some embodiments, at least a portion of the inner surfaces of the sealed chamber are mirrored. In some cases, the entirety of the inner surfaces of the sealed chamber may be mirrored.

In some embodiments, the LED array is arranged to shine light in a direction orthogonal to the plane of the one or more transparent inserts.

In further embodiments, the apparatus comprises a second LED array connected to the power source and arranged to shine light of the same frequency from an opposite side of the sealed chamber to the first LED array.

In some embodiments, the sealed chamber is a box shape, and the inlet is disposed on an opposing wall of the sealed chamber to the outlet.

In some embodiments, the power source comprises a solar panel arrangement.

In some embodiments, the apparatus comprises a pump connected to the inlet for delivering a flow of fluid containing Carbon Dioxide. The inlet may also comprise a carbonation stone for reducing the size of the CO2 bubbles of the fluid pumped into the sealed chamber.

In some embodiments, the outlet is a one-way valve.

In some embodiments, the transparent inserts are formed of a cast acrylic.

In some embodiments, the sealed chamber comprises a gasket and/or waterproof caulking for sealing the joins between its walls.

In some embodiments, the walls of the sealed chamber comprise slots for interlocking with the one or more inserts to secure them in place.

In some embodiments, the apparatus further comprises a filtration system arranged at the inlet to the sealed chamber to remove contaminants form the fluid being flowed in.

In some embodiments, the apparatus further comprises one or more sensors arranged within the sealed chamber and connected to the power source for detecting water quality of fluid being flowed in.

In such embodiments, the sensors may also be connected to a processor configured to monitor and control water concentrations via the sensors.

### BRIEF DESCRIPTION OF THE DRAWINGS

Various embodiments of the invention are disclosed in the following detailed description and accompanying drawings.

FIG. 1 illustrates an isometric view of an example bioreactor apparatus according to the present disclosure.

FIG. 2 illustrates a side view of the example bioreactor apparatus.

FIG. 3 illustrates an exploded components view of the example bioreactor apparatus.

FIG. 4 illustrates an example configuration of a one-way valve used to release oxygen gas from the sealed chamber of the bioreactor apparatus.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

The terminology used herein is for the purpose of describing particular embodiments only and is not intended to be limiting of the invention. As used herein, the term “and/or” includes any combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

An example bioreactor apparatus 100 and its components is shown in FIGS. 1-4.

Referring to FIG. 1, an isometric view of the example bioreactor apparatus 100 is shown in an assembled state. As can be seen, the apparatus comprises a sealed chamber 102 in the shape of a box with floor and ceiling elements 104 enclosing four side walls 106 (the front wall has been removed in the illustration for explanatory purposes).

The box shape is simple to construct and cheap to manufacture, as well as lending itself to a geometry that facilitates a high amount of surface area for algae growth. The chamber 102 could equally well be a different shape.

A plurality of transparent planar inserts 108 are disposed within the chamber 102, lying parallel to one another and extending across most of the width and height of the inner volume. This arrangement provides the maximum amount of surface area for algae growth.

An LED array 105 connected to a power source, for example a solar cell and battery arrangement operating at 12V and 1 Amp, is then arranged on the inner surfaces of the walls 106 facing a direction orthogonal to the plane of the inserts 108, so that the light from the LEDs shines on the algae and causes photosynthesis. Since the LED array requires such little power, the device can be entirely self-powered using a solar panel arrangement. The inner surfaces of the walls 106 are also mirrored, i.e. reflective, so that both sides of the enclosed space on either side of the inserts 108 are identical to one another.

An inlet opening 110 is formed in the floor element 104 through which a water-based fluid containing CO2 will be circulated by a pump and filter arrangement. The pump and filter may be integral to the bioreactor device or provided separately. The pump arrangement may comprise a carbonation stone for reducing the size of any Carbon Dioxide bubbles that flow into the chamber, making them easier to process because reducing the size of the bubbles leads to more surface area for the algae to react with, and thus a higher rate of photosynthesis. Carbon dioxide which is diffused via carbonation stones forms bubbles smaller than 0.5 micrometers. The micro carbon dioxide bubbles can more easily react with the and water to produce oxygen.

A gasket 112 surrounds the joint between the side walls 106 and the floor element 104 and a similar arrangement may be provided for the ceiling element—the device will be filled with fluid during operation and various other waterproofing measures such as caulking and waterproof adhesive can be used at the joints to prevent leaks.

On the opposing side of the chamber 102 to the inlet 110 is an outlet pipe 114 with a one-way valve arrangement 115 for releasing oxygen bubbles that float up from the algae following photosynthesis.

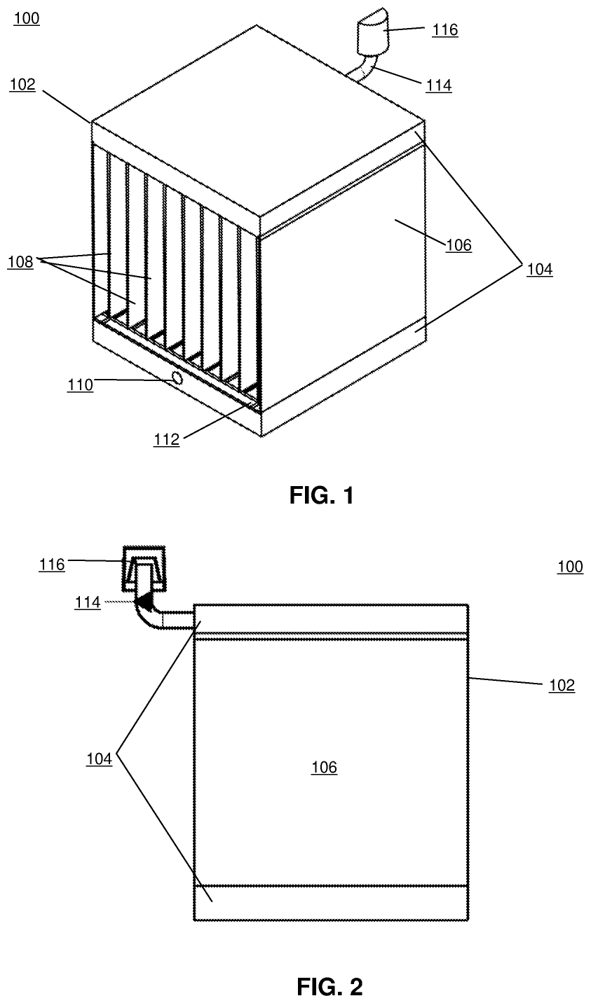

This outlet arrangement can be seen in more detail in the side-view cross-section of FIG. 2 and the close-up view of FIG. 4. The one-way valve is commonly used in brewing where brewers need to let carbon dioxide escape without letting oxygen in, but in this case is being used for the opposite purpose. Oxygen flows up the tube 114 where it bubbles into a suspended area which raises to accommodate the increase in volume. The oxygen then flows down and through the water-filled chamber and out through a set of openings. This is merely one example of a suitable valve configuration, and any number of other arrangements for achieving the same function could be used.

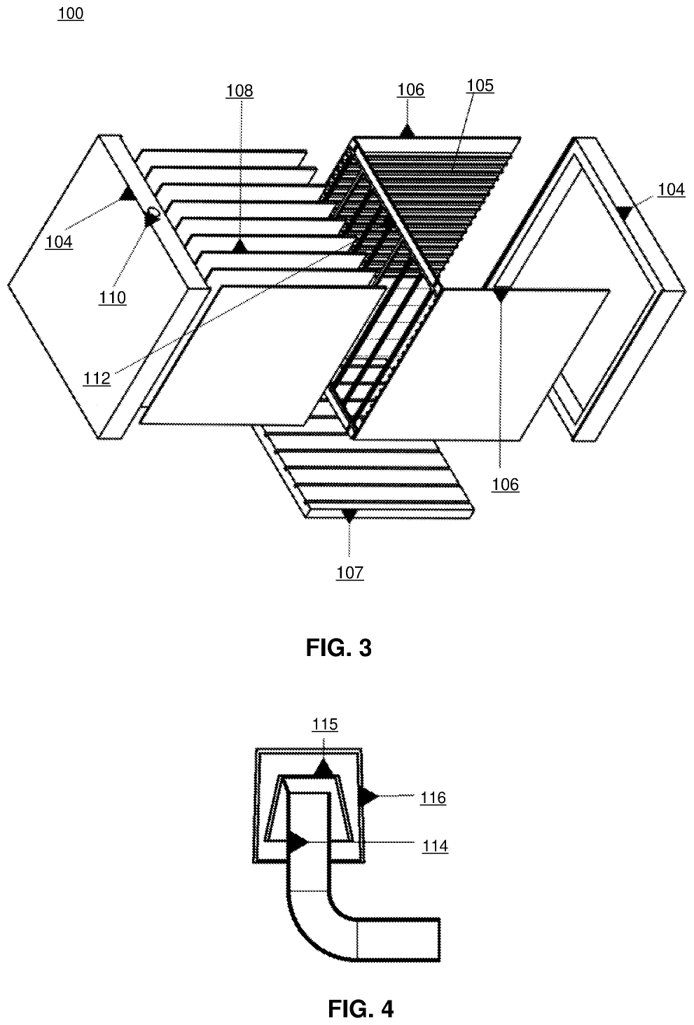

FIG. 3 illustrates an exploded components view of the example bioreactor apparatus 100, with the gasket 112 and internally disposed LED array 105 now visible. The LEDs are arranged in alternating columns, and can be placed on the inner surfaces of both walls 106.

The rear wall is also shown as having a plurality of indents 107 for securing the transparent inserts 108 in place, the four walls and the inserts are then enclosed by the hollow boxes of the floor and ceiling components 104. The construction allows for fluid exchange between different areas in the sealed chamber.

In prototype testing, it has been shown that a 1-liter version of the apparatus can be made fully functional with a 5 ml algae sample in 2-3 weeks and can produce almost 3000 lbs of oxygen a year, which is equivalent to 100 trees. The actual design is envisioned to hold 10-20 L of algae, and will photosynthesise the same amount of CO2 and produce as much oxygen as 20,000 trees at a tiny fraction of the cost and time investment. The design is relatively condensed and requires minimal maintenance, which allows for streamlined use with minimal intervention.

In order to create a living colony of algae, a tank was filled with distilled water and Cholera Vulgaris.

Specifically, the following steps were performed to obtain suitable Chlorella algae:
Pour approximately 995 mL of distilled filtered water into one glass tank with dimensions 12×12×12 cm.
Add 15 mL vial of a starter colony of Cholera Vulgaris into the glass tank.
Using a serving spoon lightly mix the solution in 5 clockwise circles and 5 counter-clockwise circles.
Place the tank in the sunlight in order for the algae to fully develop. The tanks should remain undisturbed for a week or until a sufficiently large colony of algae forms within.

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the apparatus and methods have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A bioreactor apparatus for converting Carbon Dioxide to Oxygen, the apparatus comprising:
a sealed chamber, the chamber comprising one or more walls having one or more inner surfaces;
one or more planar transparent inserts disposed within the sealed chamber, the surfaces of the one or more inserts being suitable for hosting algae;
an LED array disposed on a portion of the one or more inner surfaces of the sealed chamber, the LED array being configured to emit light suitable for causing photosynthesis in algae;
a power source connected to the LED array;
an inlet configured to deliver a flow of fluid containing Carbon Dioxide into the chamber; and
an outlet configured to release Oxygen from the sealed chamber.

2. An apparatus according to claim 1, wherein at least a portion of the inner surfaces of the sealed chamber is mirrored.

3. An apparatus according to claim 2, wherein the entirety of the inner surfaces of the sealed chamber are mirrored.

4. An apparatus according to claim 1, wherein the LED array is arranged to shine light in a direction orthogonal to the plane of the one or more transparent inserts.

5. An apparatus according to claim 1, wherein the apparatus comprises a second LED array connected to the power source and arranged to shine light of the same frequency from an opposite side of the sealed chamber to the first LED array.

6. An apparatus according to claim 1, wherein the sealed chamber is a box shape, and the inlet is disposed on an opposing wall of the sealed chamber to the outlet.

7. An apparatus according to claim 1, wherein the power source comprises a solar panel arrangement.

8. An apparatus according to claim 1, wherein the apparatus comprises a pump connected to the inlet for delivering a flow of fluid containing Carbon Dioxide.

9. An apparatus according to claim 8, wherein inlet comprises a carbonation stone for reducing the size of the CO2 bubbles of the fluid pumped into the sealed chamber.

10. An apparatus according to claim 1, wherein the outlet is a one-way valve.

11. An apparatus according to claim 1, wherein the transparent inserts are formed of a cast acrylic.

12. An apparatus according to claim 1, wherein the sealed chamber comprises a gasket and/or waterproof caulking for sealing the joins between its walls.

13. An apparatus according to claim 1, wherein the walls of the sealed chamber comprise slots for interlocking with the one or more inserts to secure them in place.

14. An apparatus according to claim 1, wherein the apparatus further comprises a filtration system arranged at the inlet to the sealed chamber to remove contaminants form the fluid being flowed in.

15. An apparatus according to claim 1, wherein the apparatus further comprises one or more sensors arranged within the sealed chamber and connected to the power source for detecting water quality of fluid being flowed in.

16. An apparatus according to claim 15, wherein the sensors may also be connected to a processor configured to monitor and control water concentrations via the sensors.
